(19) United States
(12) Patent Application Publication
(10) Pub. No.: US 2024/0423803 A1
(43) Pub. Date: Dec. 26, 2024

(54) INTRA-OPERATIVELY FITTED ANTIBIOTIC SPACER SYSTEM

(71) Applicant: THE BOARD OF REGENTS OF THE UNIVERSITY OF TEXAS SYSTEM, Austin, TX (US)

(72) Inventors: Hugh JONES, Manvel, TX (US); Ethan SIJ, Jr., Houston, TX (US); Kenneth B. MATHIS, Bellaire, TX (US)

(21) Appl. No.: 18/705,047

(22) PCT Filed: Oct. 26, 2022
(86) PCT No.: PCT/US2022/078679

§ 371 Date: Apr. 26, 2024

Related U.S. Application Data
(60) Provisional application No. 63/272,506, filed on Oct. 27, 2021.

(51) Int. Cl. A61F 2/30; A61F 2/46

(52) U.S. Cl. CPC A61F 2/30724; A61F 2002/30166; A61F 2002/30215; A61F 2002/30561; A61F 2002/30677; A61F 2002/30736; A61F 2002/30785; A61F 2002/30881; A61F 2002/30892; A61F 2002/30901; A61F 2002/4631

(57) ABSTRACT

Apparatus and methods for implanting an antibiotic spacer into a bone cavity. In certain embodiments, the antibiotic spacer comprises a shaft and a plurality of planar members coupled to the shaft. In particular embodiments, the plurality of planar members are configured such that the outer perimeter of each planar member can be intra-operatively modified.

### CROSS-REFERENCE TO RELATED APPLICATIONS

This application claims priority to U.S. Patent Application No. 63/272,506, filed Oct. 27, 2021, which is incorporated herein by reference in its entirety.

### BACKGROUND OF THE INVENTION

### Field of the Invention

Embodiments of the present invention relate to methods and apparatus for orthopedic total joint replacement. More specifically, the embodiments of the present invention relate to the revision and treatment of a total knee, hip and shoulder replacement implants due to periprosthetic joint infection.

### Background of the Related Art

Periprosthetic joint infection (PJI) following orthopedic intervention, including for example, after total knee arthroplasty (TKA) is a devastating consequence of joint arthroplasty that can result in significant bony defects. The number of periprosthetic joint infections (PJIs) continues to rise every year with as many as 75,000 estimated for 2020 alone. The economic burden of PJIs on the healthcare system was calculated to be about $785 million in 2010 alone and is projected to exceed as much as $1.5 billion in the years to come. Additionally, not only is the mortality (24% at 4.5-year follow-up) significant, but following these intrusive procedures, patients continue to live with diminished function due to loss of tissue and bone and a significant risk of recurrence of infection.

Two-stage exchange arthroplasty is the gold standard in the treatment of PJI of the knee, hip and shoulder. This method utilizes an antibiotic spacer as the intermediate stage to locally treat the infection with antibiotic-loaded polymethylmethacrylate (PMMA) bone cement and subsequent IV antibiotics for a period of about 6 weeks. [2] Exemplary embodiments create a novel antibiotic spacer to address PJI while concurrently preserving tissue and functionality.

Infection is one of the most devastating complications following total joint replacement. Although relatively uncommon, periprosthetic infections are often treated using a “two-stage” approach. Initially, the infected implant and surrounding tissue is removed and replaced with a temporary implant molded out of acrylic cement putty that has been mixed with antibiotics selected to treat the infective organism(s). This molded implant is often referred to as an “antibiotic spacer” and may be internally reinforced with metal rods or an undersized metal prosthesis to increase its strength and durability and improve the range of motion of the artificial joint. Once radiological and laboratory studies indicate that the infection has been cured (typically 6-12 weeks), the patient undergoes a second surgical procedure in which the temporary antibiotic spacer is replaced by a permanent prosthesis. Current designs of antibiotic spacers are either prefabricated under carefully regulated manufacturing conditions, or fabricated intraoperatively by the surgeon. Spacers created intraoperatively may be fabricated using pre-shaped cement molds, or fashioned and shaped in a free-hand manner around a metal rod.

Existing antibiotic spacers are typically intended to be temporary. The surgeon typically fashions and shapes the antibiotic loaded cement around the implant to obtain a best guess fit. If too much bone cement is applied, the excess amount could penetrate deep into the trabecular bone. This results in an interdigitated mass of cement and bone which can only be converted to a permanent device with significant bone loss. On the other hand, too little bone cement results in relative motion between the bone and the implant, implant migration, and pain.

### SUMMARY OF THE INVENTION

Exemplary embodiments of the present disclosure include apparatus and methods utilizing an intraoperative shapeable antibiotic delivery system comprising a defect filling total joint prosthesis designed to allow articulation of a resected joint while delivering antibiotics to infected tissues. Exemplary embodiments can be implanted in an infected joint temporarily with antibiotic-loaded bone cement and can incorporate an articular surface and a structurally supportive intramedullary rod for functionality.

Exemplary embodiments comprise modular, shapeable features located below the articular surface give the surgeon options for filling underlying bone defects. For a sizable defect, exemplary embodiments can be utilized with highly porous metaphyseal augments (e.g. cones) that are filled with antibiotic cement using a polished mold and an internal insert form. The augment can be implanted with existing instrumentation and can be formed in different size options. Once the metaphyseal augment has been implanted, the articulating surface and stem construct can be put in place within the inner cone.

In certain embodiments, the apparatus can comprise an alloy base plate that accepts a poly articulating surface, an alloy stem, and a series equidistantly spaced perpendicular planar members (e.g. fins or flanges) that are present from the base plate to the interchangeable stem tip. The spaces between the planar members can be packed with antibiotic loaded bone cement in surgery and provide increased surface area for leaching of the antibiotic for treating the periprosthetic infection. The planar members can be fabricated in size and shape that match the large pore augment's inner geometry.

In particular embodiments, the upper most planar members are slidable to allow the implant to be off centered if needed. The planar members can be manufactured from implantable polymeric materials (e.g. polyethylene) to also allow trimming and modification of the dimensions of the device intraoperatively to match the shape of the periphery of the bone cavity or defect that is presented at surgery after removal of the infected bone and prosthesis should the surgeon wish to preserve bone and not use the large pore augment. In exemplary embodiments, the interference fit of the planar members provide increased stability of the implant intraoperatively during the period of cement hardening which is often prolonged due to the effect of the antibiotic addition.

The intraoperative trimmable fin feature of this device allows the deliberate shaping of the bone/cement interface at each level by giving the surgeon a guide for cement application. Once the fins have been cut, the surgeon can place the unfilled device implant within the prepared bony cavity to test its fit. After the surgeon is satisfied with the fit, bone cement can be packed in the space between each pair of flanges until the cemented space is tangent to the peripheral edge of the fins. This helps translate the intended shape that would fill the bone defect while limiting excess cement which could interdigitate too far within the trabecular bone. Additionally, the cut fins of each level offer press-fit stabilization of the implant while the cement is curing as cement cure time can become extended when antibiotics are mixed in. In more severe cases, large amounts of trabecular bone is pulled out along with the primary infected implant. These cases would call for an augment to fill the large cavity left behind after extraction. Our metaphyseal cones can be implanted using existing revision instrumentation. As the inner geometry of the cone matches the external geometry of our antibiotic spacer stem construct, no trimming is necessary for assembly. Because of the poly articulating surface and the intimate fit achieved with both configurations, the surgeon may choose to use the implant as a one-stage revision should radiologic indications look favorable after the infection has cleared.

Exemplary embodiments of the present disclosure provide significant benefits over existing systems. For example, the intraoperative modification by trimming is intended to customize the external shape of each device according to the geometry of the defect presented intraoperatively. Exemplary embodiments of the present disclosure also offer a means for the surgeon to check the fit prior to cementing as the combined fins are essentially a skeletonized version of the final implant shape. The trimmed fins stabilize the implant and maintain its position during cement curing whereas typical existing systems have a risk of subsiding if the joint is inadvertently manipulated prior to hardening of the cement. Alternatively, the bone itself can be shaped with instrumentation designed to prepare the implantation site for placement of existing metaphyseal augments or cones. The features of exemplary embodiments of the preset disclosure also provide a higher likelihood of using the implant as a one-stage revision as opposed to other systems that are intended to be part of a two stage remedy only.

Exemplary embodiments of the present disclosure address bone defects by providing a weight-bearing articular surface, as well as a means to retain antibiotic loaded bone cement. In particular, exemplary embodiments provide the surgeon a guide for shaping PMMA bone cement into the geometries of existing metaphyseal cone augments and instrumentation increasing its potential to be a one-stage revision.

In addition, exemplary embodiments provide the ability to customize the 3D geometry of the applied bone cement to match the bone defect intraoperatively, allowing the implant to maintain its intended position while the PMMA bone cement is curing. Exemplary embodiments are also able to bare body weight without subsiding.

Certain embodiments include a modifiable antibiotic spacer apparatus comprising a shaft and a plurality of planar members coupled to the shaft, where: each planar member of the plurality of planar members is approximately perpendicular to the shaft; each planar member comprises an outer perimeter; and the plurality of planar members are configured such that the outer perimeter of each planar member can be intra-operatively modified.

Particular embodiments further comprise antibiotic cement between a first planar member of the plurality of planar members and a second planar member of the plurality of planar members. In some embodiments the plurality of planar members are configured such that the outer perimeter of each planar member can be intra-operatively modified to correspond to a prepared bone cavity created after removal of infected bony material. In certain embodiments, the prepared bone cavity is in a bone proximal to a knee, a hip or a shoulder joint. For example, in particular embodiments, the prepared bone cavity is in a femur, tibia or humerus bone. In certain embodiments the spacer apparatus can be inserted into a bone cavity in a tibia or a distal end of a femur (e.g. for a knee replacement). In some embodiments, the spacer apparatus can be inserted into a bone cavity in a proximal end of a femur (e.g. for a hip replacement), and in certain embodiments, the spacer apparatus can be inserted into a bone cavity in a humerus (e.g. for a shoulder replacement) In specific embodiments the at least one planar member of the plurality of planar members comprises an aperture extending through the at least one planar member.

Certain embodiments further comprise antibiotic cement proximal to the at least one planar member of the plurality of planar members comprising the plurality of apertures extending through the at least one planar member. In particular embodiments the aperture is configured to allow the antibiotic cement to flow through the aperture. In some embodiments at least one planar member of the plurality of planar members comprises one or more slots proximal to the shaft. In specific embodiments the one or more slots proximal to the shaft allow at least one planar member to move laterally with respect to the shaft.

In certain embodiments each planar member of the plurality of planar members comprises an implantable polymeric material. In particular embodiments each planar member of the plurality of planar members comprises polyethylene. Some embodiments further comprise a base plate configured to engage an articular surface. Specific embodiments further comprise an augment configured to receive the plurality of planar members, and in certain embodiments the augment comprises a plurality of apertures. In particular embodiments the plurality of apertures have a diameter of at least 5 mm.

In some embodiments the augment is formed from antibiotic laced polymer material, a silver coated material, tantalum or titanium. Specific embodiments further comprise an insert and a receptacle, where the insert is configured to direct bone cement through the plurality of apertures of the augment.

Certain embodiments include a method of implanting an antibiotic spacer into a bone cavity, where the method comprises: modifying dimensions of the antibiotic spacer to correspond to dimensions of the bone cavity; inserting the antibiotic spacer into the bone cavity to confirm fitment of the antibiotic spacer in the bone cavity; removing the antibiotic spacer from the bone cavity; applying an antibiotic cement to the antibiotic spacer; and inserting the antibiotic spacer into the bone cavity.

In particular embodiments the antibiotic spacer comprises a shaft and a plurality of planar members coupled to the shaft, where: each planar member of the plurality of planar members is approximately perpendicular to the shaft; and each planar member comprises an outer perimeter; and modifying dimensions of the antibiotic spacer to correspond to dimensions of the bone cavity comprises modifying the outer perimeter of at least one planar member of the plurality of planar members.

In some embodiments modifying the outer perimeter of at least one planar member of the plurality of planar members comprises trimming material from the at least one planar member of the plurality of planar members. In specific embodiments, the bone cavity is in a bone proximal or distal to a knee, a hip or a shoulder.

Certain embodiments include a method of implanting an antibiotic spacer into a bone cavity, where the method comprises: modifying dimensions of the bone cavity to correspond to dimensions of an augment; inserting the augment into the bone cavity to confirm fitment of the antibiotic spacer in the bone cavity; applying an antibiotic cement to the augment; applying antibiotic cement to an antibiotic spacer; and inserting the antibiotic spacer into the augment, where the augment comprises a plurality of apertures with a diameter of at least 5 mm.

In particular embodiments applying an antibiotic cement to the augment comprises: removing the augment from the bone cavity; placing antibiotic within the augment; directing an insert into the augment; and forcing cement through the plurality of apertures in the augment. In certain embodiments, the bone cavity is in a femur, tibia or humerus bone.

Other objects, features and advantages of the present invention will become apparent from the following detailed description, including the attached Appendix. It should be understood, however, that the detailed description and the specific examples, while indicating certain embodiments of the invention, are given by way of illustration only, since various changes and modifications within the spirit and scope of the invention will become apparent to those skilled in the art from this detailed description.

### BRIEF DESCRIPTION OF THE DRAWINGS

The objects and advantages of the present methods, system and apparatus will be better understood and more readily apparent when considered in conjunction with the following detailed description and accompanying drawings which illustrate, by way of example, preferred embodiments of this system and methods.

FIG. 1 is a front view of an apparatus according to an exemplary embodiment of the present disclosure.

FIG. 2 is a top view of the embodiment of FIG. 1 modified for insertion into a bone cavity.

FIG. 3 is a front view of the embodiment of FIG. 2 modified to be inserted into the bone cavity shown in FIG. 2.

FIG. 4 is a front view of the embodiment of FIG. 3 loaded with antibiotic cement.

FIG. 5 is a top view of a planar member of the embodiment of FIG. 1 showing holes that allow the joining of each tier of bone cement.

FIG. 6 is a exploded view of an apparatus according to an exemplary embodiment of the present disclosure.

FIG. 7 is an assembled view of the embodiment of FIG. 6.

FIG. 8 is a front view of an apparatus according to an exemplary embodiment of the present disclosure.

FIG. 9 is a perspective view of an augment configured for use with apparatus according to exemplary embodiments of the present disclosure.

FIG. 10 is a top view of the embodiment of FIG. 9.

FIG. 11 is a front view of the embodiment of FIG. 9.

FIG. 12 is a side view of the embodiment of FIG. 9.

FIG. 13 is an exploded view of an apparatus for extruding cement through the embodiment of FIG. 9.

FIG. 14 includes photographs of bone cavities or defects in which an apparatus according to an exemplary embodiment of the present disclosure may be inserted.

FIG. 15 is a photograph of an anterior (e.g. front) view of a spacer apparatus according to the present disclosure comprising a polymer shaft and plurality of planar members coupled to metal rod (not visible) and base plate.

FIG. 16 is a photograph of a spacer apparatus according to the present disclosure implanted into the cadaveric tibia in an anterior (e.g. front) view.

FIG. 17 is a photograph of a posterior (e.g. back) view of a spacer apparatus according to the present disclosure comprising a polymer shaft and plurality of planar members coupled to metal rod (not visible) and base plate.

FIG. 18 is a photograph of a spacer apparatus according to the present disclosure implanted into the cadaveric tibia in a posterior (e.g. back) view.

FIG. 19 is a photograph of a lateral (e.g. side) view of a spacer apparatus according to the present disclosure comprising a polymer shaft and plurality of planar members coupled to metal rod (not visible) and base plate.

FIG. 20 is a photograph of a spacer apparatus according to the present disclosure implanted into the cadaveric tibia in a lateral (e.g. side) view.

FIG. 21 is a photograph of a cross sectional view of an exemplary embodiment according to the present disclosure secured into a bone cavity with bone cement.

FIG. 22 includes cross-sectional views of a spacer apparatus according to the present disclosure implanted into the cadaveric tibia.

### DESCRIPTION OF EXEMPLARY EMBODIMENTS

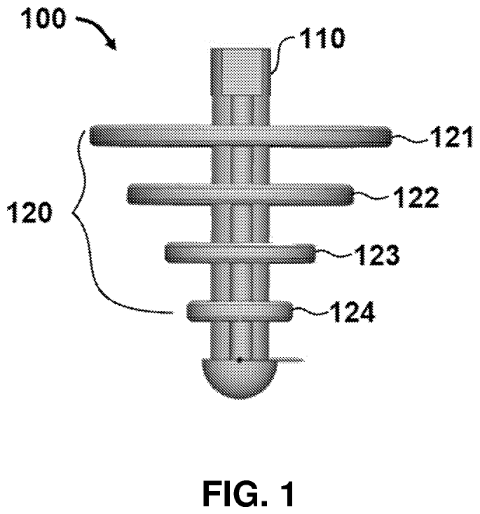

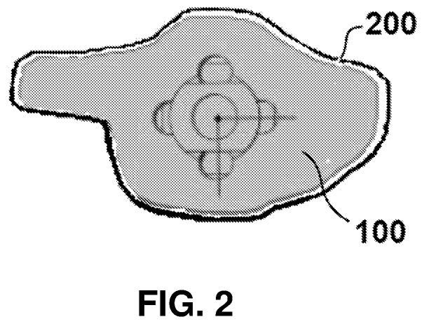

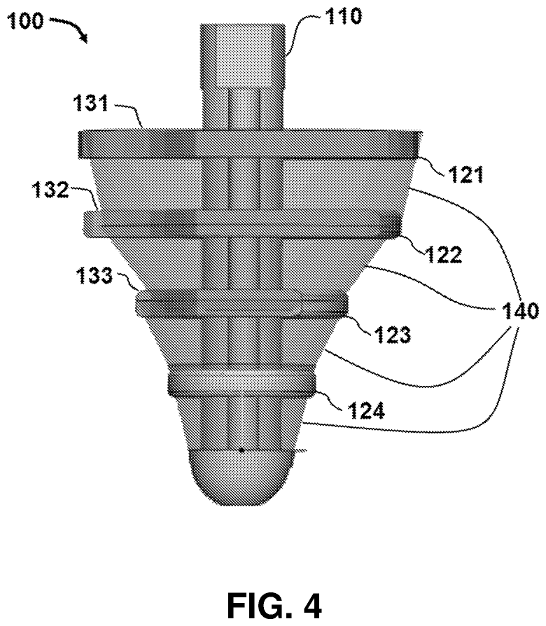

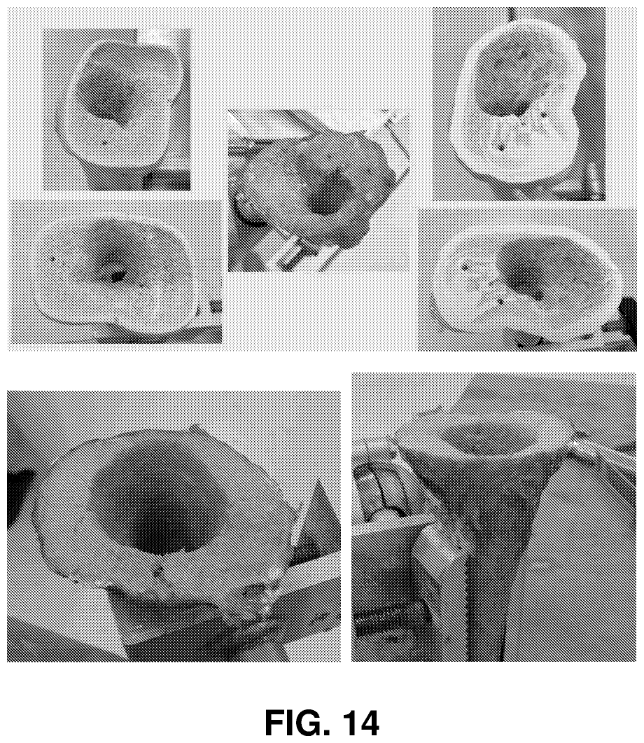

Referring initially to FIGS. 1-5, a modifiable antibiotic spacer apparatus 100 comprises a shaft 110 and a plurality of planar members 120 coupled to shaft 100. In the embodiment shown, each of the planar members 121-124 are approximately perpendicular to shaft 110. As described more fully below, the plurality of planar members 120 are configured such that the outer perimeter of each planar member 121-124 can be intra-operatively modified to correspond to a prepared bone cavity 200 (shown in FIG. 2) created after removal of infected bony material. Additional photographs of bone cavities or defects are shown in FIG. 14.

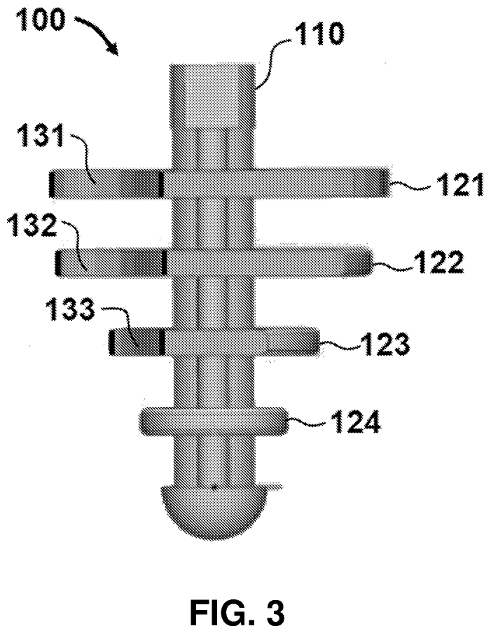

In certain embodiments, planar members 121-124 may comprise an implantable polymeric material, including for example, polyethylene, that is capable of being modified (e.g. by trimming or cutting) to correspond to dimensions of bone cavity 200. In the embodiment shown, planar members 121, 122 and 123 have each had a portion of their outer perimeters 131, 132 and 133 trimmed to correspond to the dimensions of bone cavity 200. In the views shown in FIGS. 2 and 3, the left proximal portions of outer perimeters 131, 132 and 133 have been trimmed to correspond to the shape and dimensions of bone cavity 200. In certain embodiments, planar members 121-124 may comprise slots proximal to shaft 110 to allow planar members 121-124 to move laterally with respect to shaft 110.

Implant 100 can be inserted into and removed from bone cavity 200 multiple times to allow iterations of progressive trimming and to test the fitment between apparatus 100 and bone cavity 200. When an acceptable fitment between implant 100 and bone cavity 200 has been achieved, antibiotic cement 140 can be applied to implant 100. In particular examples, antibiotic cement 140 can be applied between planar members 121-124. In specific embodiments, antibiotic cement 140 may be antibiotic-loaded polymethylmethacrylate.

In testing of embodiments according to the present disclosure a first spacer apparatus was trimmed in 8 minutes and 55 seconds, and cemented in 5 minutes and 10 seconds, for a total time of 14 minutes and 5 seconds. A second spacer apparatus was trimmed in 5 minutes and 33 seconds, and cemented in 6 minutes and 15 seconds for a total of 11 minutes and 48 seconds. The first spacer apparatus was removed in 37 seconds, while the second spacer apparatus remained implanted. The average time to trim for the two example spacer apparatus was 7 minutes and 14 seconds, and the average time to cement the two examples as 5 minutes and 42 seconds.

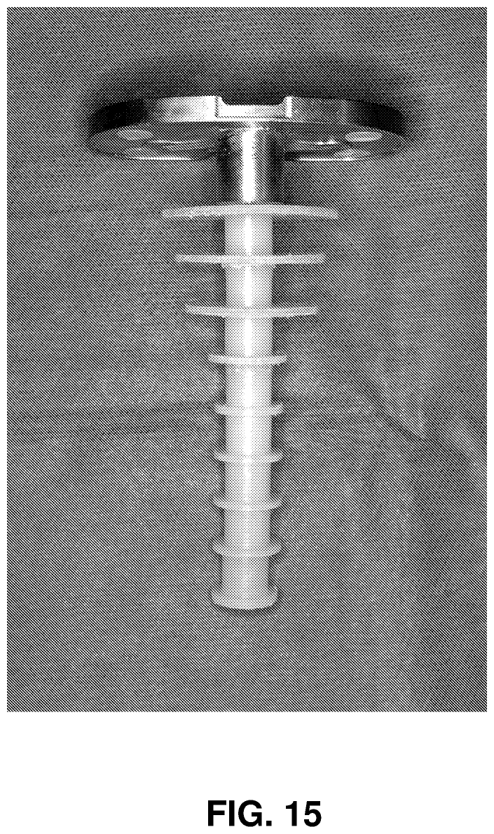

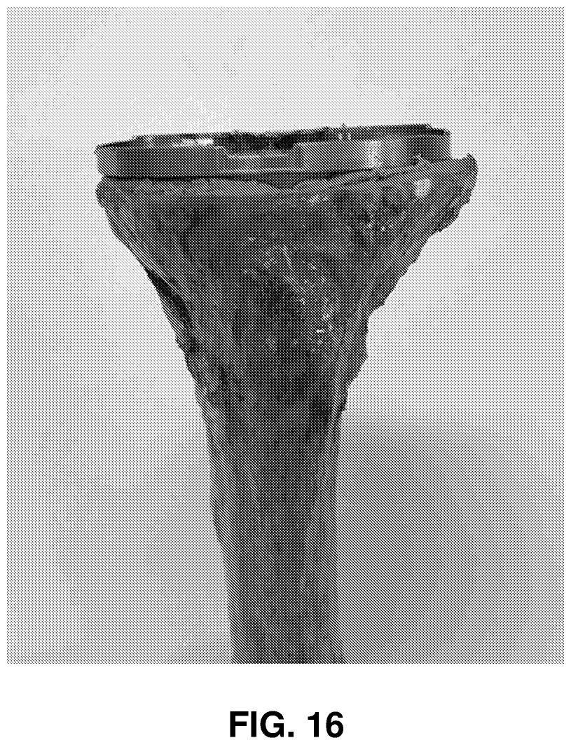

FIG. 15 is a photograph of an anterior (e.g. front) view of an exemplary embodiment according to the present disclosure comprising a polymer shaft and plurality of planar members coupled to metal rod (not visible) and base plate. FIG. 16 is a photograph of an anterior (e.g. front) view of an exemplary embodiment according to the present disclosure inserted into a bone cavity a tibia and secured with bone cement. The base plate and bone cement are visible in this view.

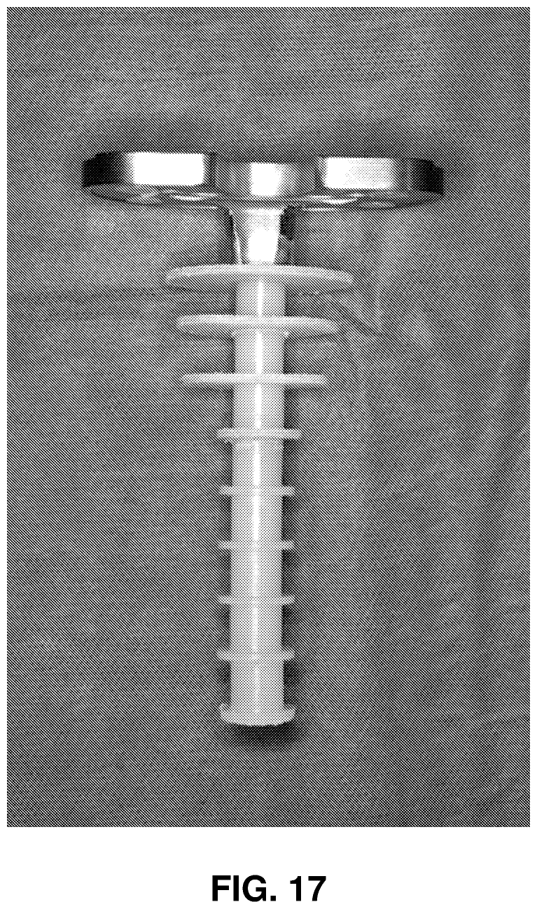

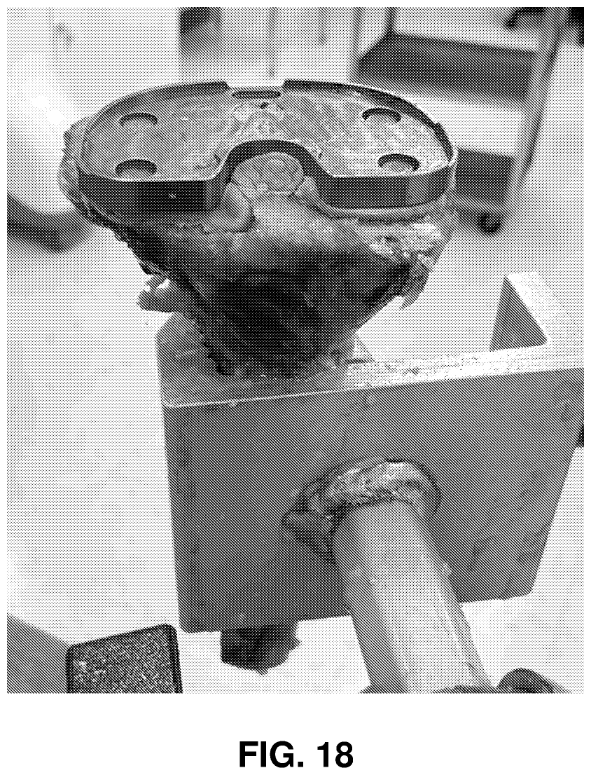

FIG. 17 is a photograph of a posterior (e.g. back) view of an exemplary embodiment according to the present disclosure comprising a polymer shaft and plurality of planar members coupled to metal rod (not visible) and base plate. FIG. 18 is a photograph of a posterior (e.g. back) view of an exemplary embodiment according to the present disclosure inserted into a bone cavity a tibia and secured with bone cement. Again, only the base plate and bone cement are visible in this view when the apparatus is inserted and secured into the bone cavity.

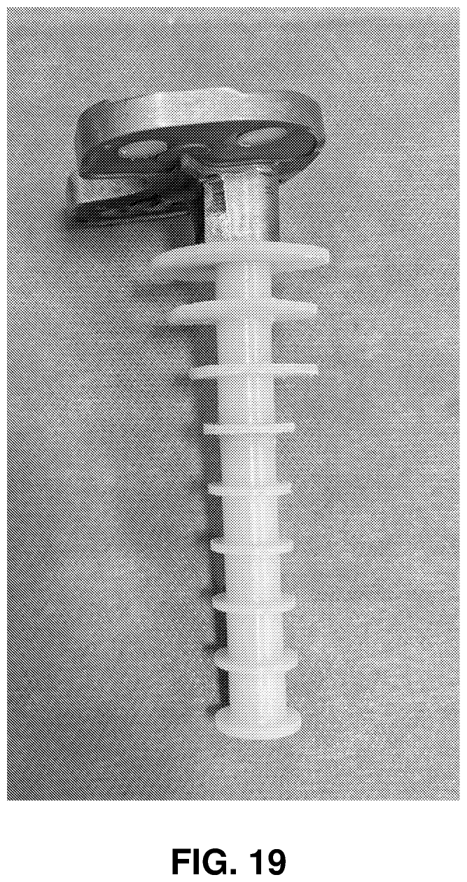

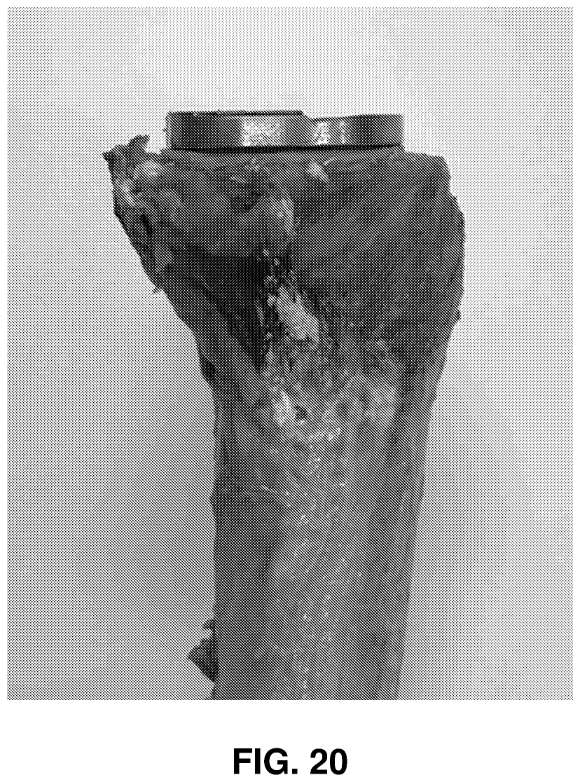

FIG. 19 is a photograph of a lateral (e.g. side) view of an exemplary embodiment according to the present disclosure comprising a polymer shaft and plurality of planar members coupled to metal rod (not visible) and base plate. FIG. 20 is a photograph of a lateral (e.g. side) view of an exemplary embodiment according to the present disclosure inserted into a bone cavity in a tibia and secured with bone cement. The base plate and bone cement are visible in this view.

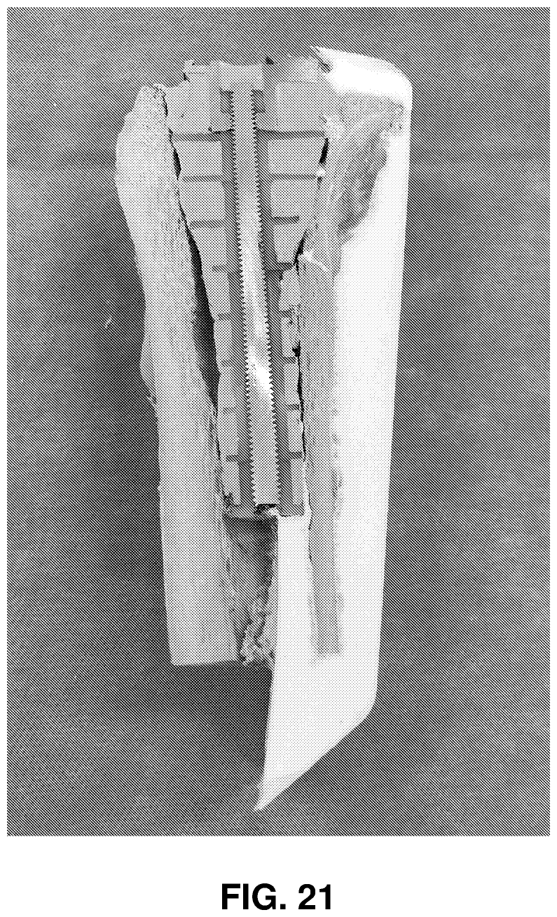

FIG. 21 is a photograph of a cross sectional view of an exemplary embodiment according to the present disclosure showing a metal (e.g. titanium) base plate and rod coupled to a polymer shaft and planar members. The apparatus has been secured into a bone cavity with bone cement.

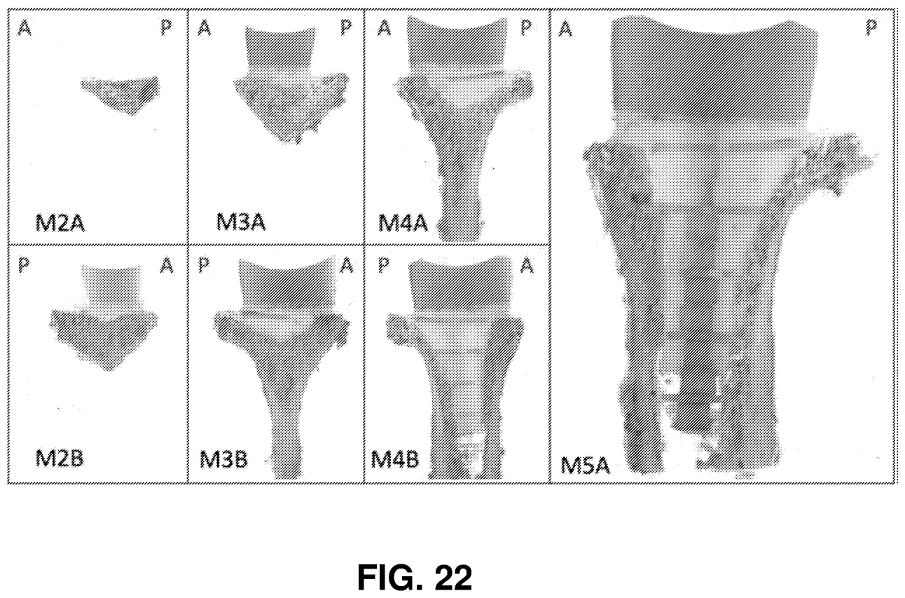

FIG. 22 illustrates 10 mm cross-section views of the medial portion of the second example spacer apparatus implanted into the cadaveric tibia. The medial 2 image section (M2) is the most medial portion of the implant while medial 5 image section (M5) is the most lateral. The “A” represents the anterior and the “P” represents the posterior. While a tibia is shown in the examples illustrated in FIGS. 15-18, it is understood that other examples may include a bone cavity in other bones, including for example a bone proximal to a knee, a hip or a shoulder.

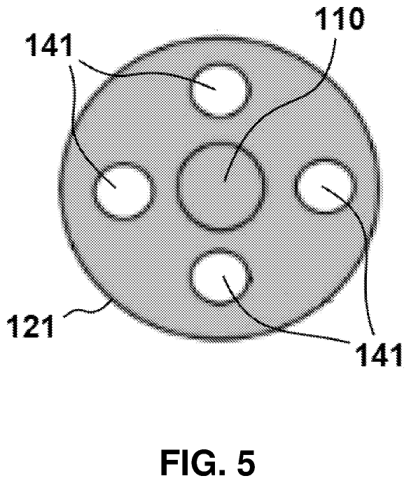

In certain embodiments, one or more of planar members 121-124 may comprise a plurality of apertures extending through the planar member to allow antibiotic cement 140 to extrude or flow through a planar member. For example in the top view shown in FIG. 5, planar member 121 comprises a plurality of apertures 141. It is understood that planar members 122-124 may also comprise apertures similar to those shown in FIG. 5. While a circular outer perimeter for planar member 121 is shown in FIG. 5, other embodiments may comprise planar members with a different configuration outer perimeter, including for example, an oval.

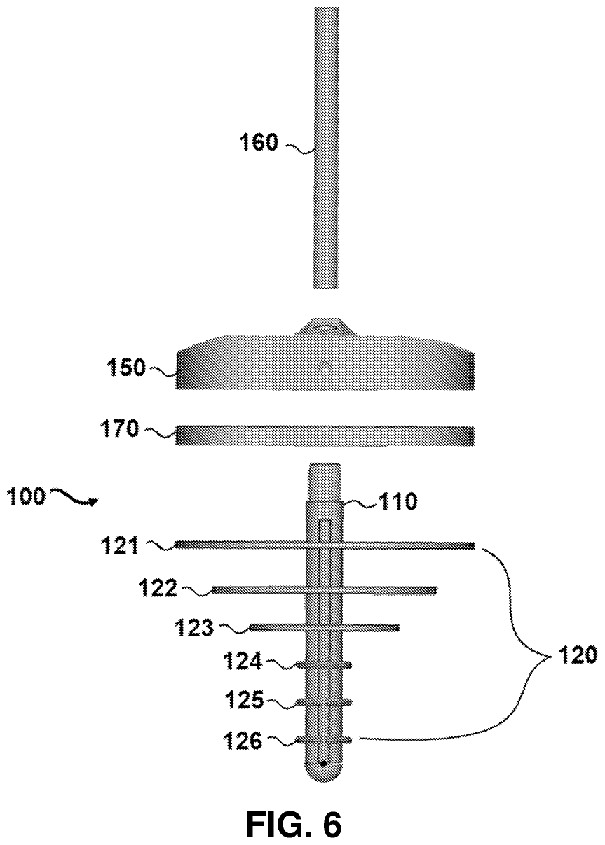

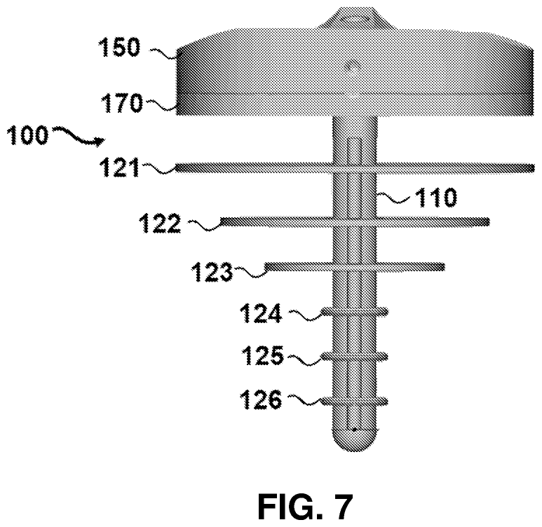

Referring now to FIGS. 6-7, in this exemplary embodiment of modifiable antibiotic spacer apparatus 100, the plurality of planar members 120 comprises individual planar members 121-126 coupled to shaft 110. An exploded view is shown in FIG. 6, while an assembly view is shown in FIG. 7. As in the previously-described embodiment, planar members 121-126 are trimmable by a surgeon or other medical professional such that apparatus 100 can be intra-operatively modified such that its shape and dimensions correspond to a bone cavity. In addition, this embodiment comprises a base plate 170 configured to engage an articular surface 150 of a prosthetic joint. In addition, apparatus 100 comprises a rod 160 configured to be inserted into shaft 110. In particular embodiments, rod 160 may be a titanium rod configured to provide structural support to shaft 110 and apparatus 100. Because of the poly articulating surface and the intimate fit achieved with embodiments disclosed herein, the surgeon may choose to use apparatus 100 as a one-stage revision should radiologic indications look favorable after the infection has cleared.

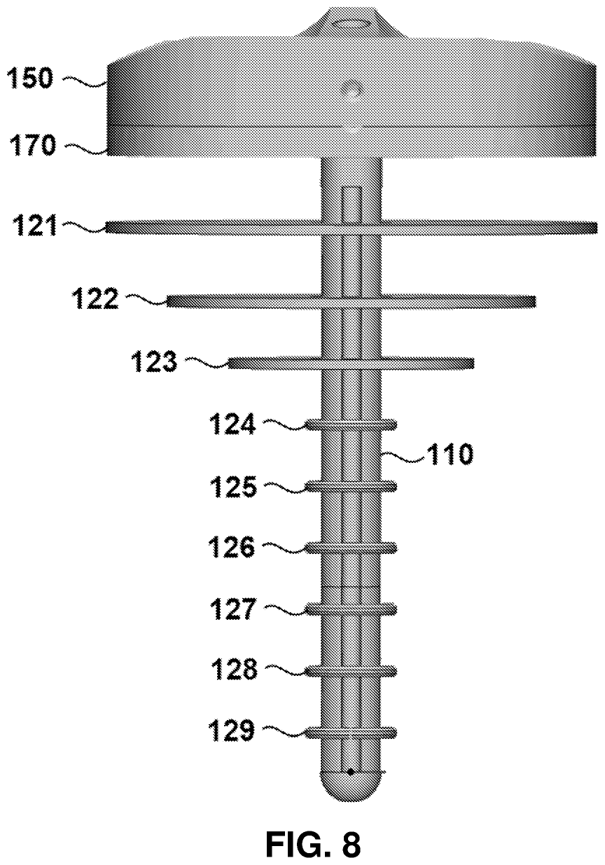

Referring now to FIG. 8, an embodiment of apparatus 100 is shown that is similar to the embodiment shown in FIGS. 6-7. In this embodiment, apparatus 100 comprises a plurality of planar members 120 with planar members 121-129 that can be intra-operatively modified to correspond to a prepared bone cavity as previously described. Exemplary embodiments of apparatus 100 may comprise different lengths of shaft 110 and/or a different number or spacing for planar members 120 to accommodate different depths into the intramedullary canal for added functional stability should the proximal bone be too deteriorated to support the implant when under load.

In severe applications, large amounts of trabecular bone may be pulled out along with the primary infected implant prior to implantation of apparatus 100. Such applications may utilize an augment to fill the large cavity left behind after extraction. In such embodiments, it may be desirable to modify a bone cavity to accept an augment to receive apparatus 100. For example, if the existing bone cavity is large in size, the cavity can be prepared to receive an adapter that can accept apparatus 100 without trimming or modification of planar members 120.

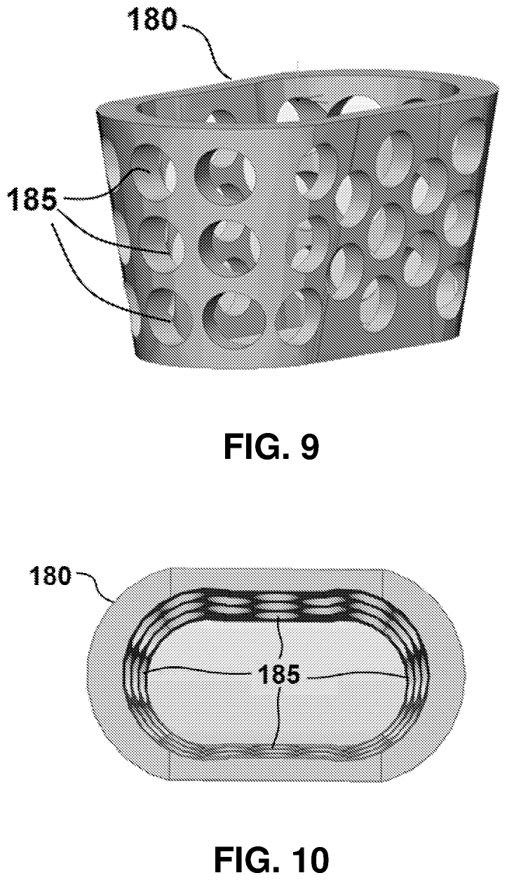

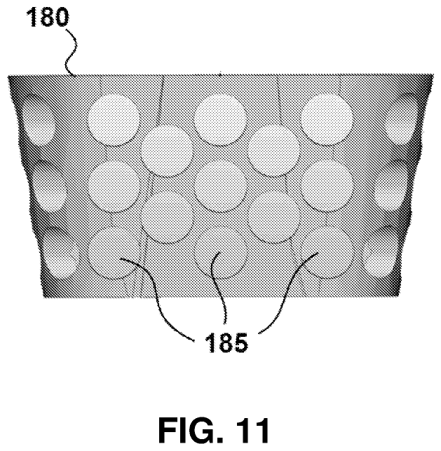

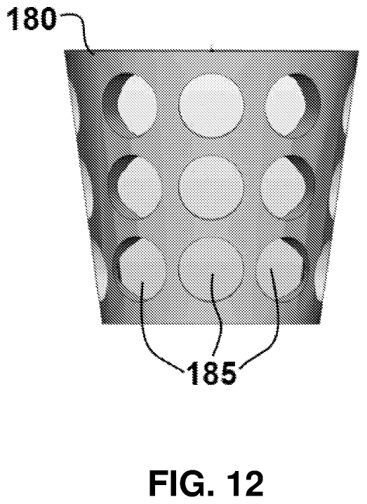

Referring now to FIGS. 9-12, an augment 180 is shown in perspective, top, front and side views respectively. In this embodiment, augment 180 is a metaphyseal augment with an oval tapered configuration, but other embodiments may include other shapes, including for example a cone. In examples incorporating augment 180, the bone cavity can be prepared using existing revision instrumentation (e.g. broaches and reamers). The inner geometry of augment 180 can be configured to match the external geometry of planar members 120, such that planar members can be received within augment 180. Accordingly, trimming of planar members 120 may not be needed for assembly.

In certain embodiments, augment 180 may comprise a plurality of apertures 185 that are sized sufficiently to allow antibiotic cement to extrude or flow through the apertures. For example, apertures 185 may be 5 mm, 6 mm, 7 mm, 8 mm, 9 mm or 10 mm in diameter in certain embodiments. Augment 180 may also be formed from suitable materials including for example, an antibiotic laced polymer material, a silver coated material, or other materials including tantalum or titanium.

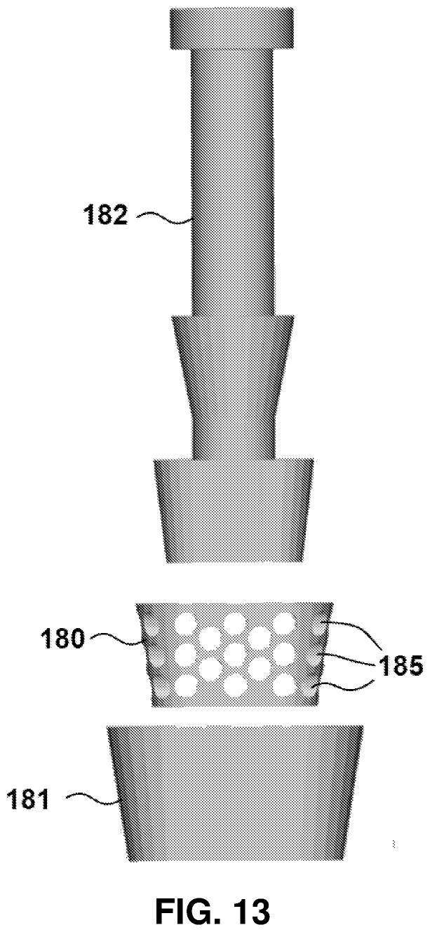

Referring now to FIG. 13, an apparatus for extruding cement through apertures 185 is shown. In this embodiment, cement can be placed within augment 180 which can in turn be placed within a receptacle 181. An insert 182 can then be directed into augment 180 to force cement through apertures 185. In exemplary embodiments, the external configuration of insert 182 corresponds to the internal configuration of augment 180. Similarly, the external configuration of augment 180 corresponds to the internal configuration of 181. Accordingly, insert 182 can be used to effectively direct cement through apertures 185. This process can be beneficial in forcing or extruding cement through apertures 185, as the viscosity of typical bone cement is quite high. Ensuring cement is directed through apertures 185 can improve bond strength between augment 180 and a bone cavity into which augment 180 is placed, as well as improve bond strength between augment 180 and apparatus 100 inserted into augment 180.

Any embodiment of any of the present apparatus, methods, composition, kit, and systems may consist of or consist essentially of—rather than comprise/include/contain/have—the described steps and/or features. Thus, in any of the claims, the term “consisting of” or “consisting essentially of” may be substituted for any of the open-ended linking verbs recited above, in order to change the scope of a given claim from what it would otherwise be using the open-ended linking verb.

The use of the term “or” in the claims is used to mean “and/or” unless explicitly indicated to refer to alternatives only or the alternatives are mutually exclusive, although the disclosure supports a definition that refers to only alternatives and “and/or.”

Throughout this application, the term “about” or “approximately” is used to indicate that a value includes the standard deviation of error for the device or method being employed to determine the value.

Following long-standing patent law, the words “a” and “an,” when used in conjunction with the word “comprising” in the claims or specification, denotes one or more, unless specifically noted.

As used herein, the terms “comprises,” “comprising,” “includes,” “including,” “has,” “having,” or any other variation thereof, are intended to cover a non-exclusive inclusion. For example, a process, product, article, or apparatus that comprises a list of elements is not necessarily limited only those elements but may include other elements not expressly listed or inherent to such process, process, article, or apparatus.

Furthermore, the term “or” as used herein is generally intended to mean “and/or” unless otherwise indicated. For example, a condition A or B is satisfied by any one of the following: A is true (or present) and B is false (or not present), A is false (or not present) and B is true (or present), and both A and B are true (or present). As used herein, including the claims that follow, a term preceded by “a” or “an” (and “the” when antecedent basis is “a” or “an”) includes both singular and plural of such term, unless clearly indicated within the claim otherwise (i.e., that the reference “a” or “an” clearly indicates only the singular or only the plural). Also, as used in the description herein, the meaning of “in” includes “in” and “on” unless the context clearly dictates otherwise.

As used herein, “intra-operatively modified” is used to describe an apparatus or component that is capable of being modified by a surgeon or other medical professional to a desired shape during a medical operation, including for example, implantation of an antibiotic spacer to address a periprosthetic joint infection.

As used herein, “patient” or “subject” includes mammalian organisms, such as human and non-human mammals, for example, but not limited to, rodents, mice, rats, non-human primates, companion animals such as dogs and cats as well as livestock, e.g., sheep, cow, horse, etc. Therefore, for example, although the described embodiments illustrate use of the present methods on humans, those of skill in the art would readily recognize that these methods and compositions could also be applied to veterinary medicine as well as on other animals.

As used herein, and unless otherwise indicated, the terms “treat,” “treating,” “treatment” and “therapy” contemplate an action that occurs while a patient is suffering from a disease or disorder that reduces the severity of one or more symptoms or effects of such disease or disorder. Where the context allows, the terms “treat,” “treating,” and “treatment” also refers to actions taken toward ensuring that individuals at increased risk of a disease or disorder, are able to receive appropriate surgical and/or other medical intervention prior to onset of a disease or disorder. As used herein, and unless otherwise indicated, the terms “prevent,” “preventing,” and “prevention” contemplate an action that occurs before a patient begins to suffer from a disease or disorder, that delays the onset of, and/or inhibits or reduces the severity of a disease or disorder.

As used herein, and unless otherwise indicated, the terms “manage,” “managing,” and “management” encompass preventing, delaying, or reducing the severity of a recurrence of a disease or disorder in a patient who has already suffered from such a disease, disorder or condition. The terms encompass modulating the threshold, development, and/or duration of a disease or disorder or changing how a patient responds to a disease or disorder.

As used herein, and unless otherwise specified, a “therapeutically effective amount” of cells, factor or compound is an amount sufficient to provide any therapeutic benefit in the treatment or management of a disease or disorder, or to delay or minimize one or more symptoms associated with a disease or disorder. A therapeutically effective amount means an amount of the cells, factor or compound, alone or in combination with one or more other therapies and/or therapeutic agents that provide any therapeutic benefit in the treatment or management of a disease or disorder. The term “therapeutically effective amount” can encompass an amount that alleviates a disease or disorder, improves or reduces a disease or disorder, improves overall therapy, or enhances the therapeutic efficacy of another therapeutic agent.

The corresponding structures, materials, acts, and equivalents of all means or steps plus function elements in the claims below are intended to include any structure, material, or act for performing the function in combination with other claimed elements as specifically claimed. The description of the present invention has been presented for purposes of illustration and description, but it is not intended to be exhaustive or limited to the invention in the form disclosed. Many modifications and variations will be apparent to those of ordinary skill in the art without departing from the scope and spirit of the invention. The embodiment was chosen and described in order to best explain the principles of the invention and the practical application, and to enable others of ordinary skill in the art to understand the invention for various embodiments with various modifications as are suited to the particular use contemplated.

Without further elaboration, it is believed that one skilled in the art can, using the description herein, utilize the present methods to its fullest extent. The embodiments described herein are to be construed as illustrative and not as constraining the remainder of the disclosure in any way whatsoever. While preferred embodiments have been shown and described, many variations and modifications thereof can be made by one skilled in the art without departing from the spirit and teachings of the presently disclosed methods.

Accordingly, the scope of protection is not limited by the description set out above, but is only limited by the claims, including all equivalents of the subject matter of the claims. The disclosures of all patents, patent applications and publications cited herein are hereby incorporated herein by reference, to the extent that they are consistent with the present disclosure set forth herein.

### REFERENCES

The following references, to the extent that they provide exemplary procedural or other details supplementary to those set forth herein, are specifically incorporated herein by reference.
1 1. Kurtz S M et al. J Arthroplasty. 2012 September; 27 (8 Suppl): 61-5.e1. doi: 10.1016/j.arth.2012.02.022. Epub 2012 May 2. PMID: 22554729.
2. Tande A J et al. Clin Microbiol Rev. 2014 April; 27 (2): 302-45. doi: 10.1128/CMR.00111-13. PMID: 24696437; PMCID: PMC3993098.
3. Engh G. A. In: Scuderi G. R., Tria A. J. (eds) Surgical Techniques in Total Knee Arthroplasty. Springer, New York, NY; https://doi.org/10.1007/0-387-21714-2_53.

We claim:

1. A modifiable antibiotic spacer apparatus comprising:
a shaft; and
a plurality of planar members coupled to the shaft, wherein:
each planar member of the plurality of planar members is approximately perpendicular to the shaft;
each planar member comprises an outer perimeter; and
the plurality of planar members are configured such that the outer perimeter of each planar member can be intra-operatively modified.

2. The modifiable antibiotic spacer apparatus of claim 1 further comprising antibiotic cement between a first planar member of the plurality of planar members and a second planar member of the plurality of planar members.

3. The modifiable antibiotic spacer apparatus of claim 1 or 2 wherein the plurality of planar members are configured such that the outer perimeter of each planar member can be intra-operatively modified to correspond to a prepared bone cavity created after removal of infected bony material.

4. The modifiable antibiotic spacer apparatus of claim 3 wherein the plurality of planar members are configured such that the outer perimeter of each planar member can be intra-operatively modified to correspond to the prepared bone cavity, and wherein the prepared bone cavity is in a femur, tibia or humerus bone.

5. The modifiable antibiotic spacer apparatus of any of claims 1-4 wherein at least one planar member of the plurality of planar members comprises an aperture extending through the at least one planar member.

6. The modifiable antibiotic spacer apparatus of claim 5 further comprising antibiotic cement proximal to the at least one planar member of the plurality of planar members comprising the plurality of apertures extending through the at least one planar member.

7. The modifiable antibiotic spacer apparatus of claim 5 wherein the aperture is configured to allow the antibiotic cement to flow through the aperture.

8. The modifiable antibiotic spacer apparatus of any one of claims 1-7 wherein at least one planar member of the plurality of planar members comprises one or more slots proximal to the shaft.

9. The modifiable antibiotic spacer apparatus of claim 8 wherein the one or more slots proximal to the shaft allow the at least one planar member to move laterally with respect to the shaft.

10. The modifiable antibiotic spacer apparatus of any one of claims 1-9 wherein each planar member of the plurality of planar members comprises an implantable polymeric material.

11. The modifiable antibiotic spacer apparatus of any one of claims 1-10 wherein each planar member of the plurality of planar members comprises polyethylene.

12. The modifiable antibiotic spacer apparatus of any one of claims 1-11 further comprising a base plate configured to engage an articular surface.

13. The modifiable antibiotic spacer apparatus of any one of claims 1-11 further comprising an augment configured to receive the plurality of planar members.

14. The modifiable antibiotic spacer apparatus of claim 13 wherein the augment comprises a plurality of apertures.

15. The modifiable antibiotic spacer apparatus of claim 14 wherein the plurality of apertures have a diameter of at least 5 mm.

16. The modifiable antibiotic spacer apparatus of any one of claims 13-15 wherein the augment is formed from antibiotic laced polymer material, a silver coated material, tantalum or titanium.

17. The modifiable antibiotic spacer apparatus of any one of claims 13-16 further comprising an insert and a receptacle, wherein the insert is configured to direct bone cement through the plurality of apertures of the augment.

18. A method of implanting an antibiotic spacer into a bone cavity, the method comprising:
modifying dimensions of the antibiotic spacer to correspond to dimensions of the bone cavity;
inserting the antibiotic spacer into the bone cavity to confirm fitment of the antibiotic spacer in the bone cavity;
removing the antibiotic spacer from the bone cavity;
applying an antibiotic cement to the antibiotic spacer; and
inserting the antibiotic spacer into the bone cavity.

19. The method of claim 18 wherein:
the antibiotic spacer comprises:
a shaft; and
a plurality of planar members coupled to the shaft, wherein:
each planar member of the plurality of planar members is approximately perpendicular to the shaft; and
each planar member comprises an outer perimeter; and
modifying dimensions of the antibiotic spacer to correspond to dimensions of the bone cavity comprises modifying the outer perimeter of at least one planar member of the plurality of planar members.

20. The method of claim 18 or 19 wherein modifying the outer perimeter of at least one planar member of the plurality of planar members comprises trimming material from the at least one planar member of the plurality of planar members.

21. The method of any one of claims 18-20 wherein the bone cavity is in a femur, tibia or humerus bone.

22. A method of implanting an antibiotic spacer into a bone cavity, the method comprising:
modifying dimensions of the bone cavity to correspond to dimensions of an augment;
inserting the augment into the bone cavity to confirm fitment of the antibiotic spacer in the bone cavity;
applying an antibiotic cement to the augment;
applying antibiotic cement to an antibiotic spacer; and
inserting the antibiotic spacer into the augment, wherein:
the augment comprises a plurality of apertures with a diameter of at least 5 mm.

23. The method of claim 22, wherein applying an antibiotic cement to the augment comprises:
removing the augment from the bone cavity;
placing antibiotic within the augment;
directing an insert into the augment; and
forcing cement through the plurality of apertures in the augment.

24. The method of claim 22 or claim 23 wherein the bone cavity is in a femur, tibia or humerus bone.
